(19) United States
(12) Patent Application Publication
(10) Pub. No.: US 2004/0260066 A1
(43) Pub. Date: Dec. 23, 2004

(54) Method for purifying mixtures of immunoglobulin & albumin

(76) Inventors: Juan Felipe de Leon, Cordoba (AR); Domingo Antonio Murature, Cordoba (AR); Hilda Lidia Nieres, Cordoba (AR)

Correspondence Address: John H. Faro, Esq., Faro & Associates, P.O. Box 4904, Key Biscayne, FL, 33149-4904, US

(21) Appl. No.: 10/465,672

(22) Filed: Jun. 20, 2003

(51) Int. Cl. C07K 16/18; C07K 14/765

(52) U.S. Cl. 530/363; 530/387.1

(57) ABSTRACT

A method for the concurrent purification and isolation of albumin and IgG from a fluid composition containing up to about 15%, by weight, of blood plasma proteins obtained from a plasma source that has not previously undergone any prior fractionation. In this method, the composition is buffered and thereafter modified by the addition, relative to IgG, of a thermal stabilization effective amount of a polyhydric alcohol, and, by addition, relative to albumin, of a thermal stabilization effective amount of a carboxylic acid (e.g. carboxylic acid having about 3 to about 10 carbon atoms), or the physiologically acceptable salt of said. The resulting fluid composition is thereafter heated to and maintained at a temperature in the range sufficient to effect essentially complete denaturation of proteins, other than the albumin and the IgG components, of said composition. Thereafter, the fluid composition is cooled and the soluble components thereof, the albumin and IgG, isolated from the solids and suspended matter by a combination of filters designed to initially remove the solids and suspended matter from the fluid composition, and thereafter through an array of tangential flow filtration membranes capable of isolation of soluble protein components of said composition by molecular weight.

### BACKGROUND OF THE INVENTION

1. Field of the Invention

This invention relates to a method and to the product obtained thereby. More specifically, this invention is directed to a method for producing essentially purified protein fractions from a heterogeneous mixture, wherein the mixture includes proteinacious materials that are both endogenous to biological fluids, (such as plasma) and exogenous to such fluids (e.g. pathogens such as viruses and their by-products). The method of this invention enables the concurrent attainment of highly purified albumin and human immunoglobulin (IgG) fractions directly from plasma, without prior fractionation of the plasma, so as to produce higher yields of therapeutically active proteins than attainable by previous methods. The albumin attainable in accordance with the method of this invention is useful, for example, in so called “replacement solutions” to compensate for blood losses. Moreover, the immunoglobulin fraction attainable in accordance with the method of this invention is effective in the prevention and treatment of a number of infectious diseases, including those associated with Staphylococci, Streptococci, Coli, Pseudomonas, Herpes zoster and pyocyaneus septicemias.

2. Description of the Prior Art

The use of biological products derived from biological fluids has and continues to present both serious concern and challenges relative to contamination by both exogenous agents and endogenous materials that maybe transmissive of various diseases and infections. Accordingly, there has and continues to be increasing diligence in the screening of blood donors by blood banks; and, vigilance in the purification of biological products, derived from blood, to eliminate undesirable materials that can transmit infectious agents to the recipient thereof. Unfortunately, since a number of the infectious agents found in blood are physiologically and chemically similar to the desirable blood products, the processes used in the separation or inactivation such agents can potentially destroy or reduce the therapeutic properties of the desirable blood components. Thus, many of the generally accepted processes must sacrifice efficiency, and accordingly yields, to insure the preservation of the therapeutic activity of the commercially valuable protein products they seek to purify and isolate from biological fluids, such as blood and its component fractions.

Notwithstanding such concerns, many useful blood fractions and blood proteins are obtained from human blood plasma by fractionation according to known techniques such as, for example, the alcohol fractionation method of Cohn described in U.S. Pat. No. 2,390,074 (1945) and the Journal of the American Chemical Society, Vol. 68, page 459 (1946) and the Rivanol, RTM ammonium sulfate method. Each of these methods, as well as other variations and techniques, are summarized in “The Plasma Proteins”, second edition, Volume III, pages 548-550, Academic Press, New York, N.Y. (1977).

Two of the more valuable blood products which have and continue to enjoy substantial efficacy and commercial value include albumin and human immunoglobulin (IgG). These two blood products are generally obtained from the plasma fraction of whole blood. In practice, when whole blood is no longer suitable for transfusion, the plasma fraction thereof for later recovery of therapeutically desirable fractions thereof. Two of the more desirable components of this plasma fraction are the albumin and the immunoglobulins present therein. As shall be evident from the discussion which follows, the isolation and concentration of these components from plasma can present formidable obstacles and challenges. At the outset it is noted each of these components are essentially protein in chemical composition, and thus sensitive to denaturation upon heating to in excess of physiological temperatures (40 to 45° C.). Moreover, because of the inherent properties of the albumin, it tends to associate itself with other plasma components, further complicating its isolation and recovery. Comparable difficulties are encountered with the plasma IgG fraction. Accordingly, methods for the isolation and recovery of albumin and IgG from human plasma have generally required some form of stabilization of such materials prior to subjecting them to purification methods relying upon elevated temperatures for denaturation of the proteins associated with infections disease states. Moreover, because of the dissimilar chemistry and structure of albumin and IgG, it has not been previously practical to attempt to isolate and recover each of these materials concurrently, thus, necessitating multiple and often complex fractionation processes.

The problems associated with the separation and recovery of selected and desirable proteins from heterogeneous fluids has been addressed in various environments and with various objectives in mind. The following patents are representative of the patent literature relating to such protein separation and recovery techniques.

U.S. Pat. No. 4,440,679 (to Fernandes, et al, issued Apr. 3, 1984) discloses a method for the pasteurization of fluid compositions containing thermally sensitive, therapeutically active proteins. According to the Fernandes method, a protein solution is rendered heat stable, during pasteurization or heating at a temperature of about 60 to 75° C., by mixing with heat-stabilizing or pasteurization-stabilizing amounts of a polyol. The term “polyol” is defined by Fernandes as a substance with more than one hydroxyl group (—OH), and includes polyhydric alcohols and carbohydrates, such as sugars. According to Fernandes, the polyols that are preferred for use in his method are water miscible, physiologically compatible with the protein, and have a low molecular weight, i.e., a molecular weight less than about 5000. These polyhydric alcohols include both simple sugars and polyhydric alcohols. Typical examples of sugars that may be used in the Fernandes method are mono-, di-, and trisaccharides such as arabinose, glucose, galactose, fructose, ribose, mannose, rhamnose, sucrose, maltose, raffinose, melezitose, and so forth. Exemplary of polyhydric alcohols or reduced sugars, included within the purview of the Fernandes invention are erythritol, ribitol, sylitol, sorbitol, mannitol, etc.

U.S. Pat. No. 4,754,019 (to Gion, et al., issued Jun. 28, 1988) discloses a method for recovering a substantially pure albumin solution from a protein solution containing albumin and other plasma proteins of human origin, by heating a buffered (pH of 4.5 to 5.5) protein solution at a temperature of 65 to 70° C., for 15 to 60 minutes, in the presence of 3 to 10 mM of an organic carboxylic acid having 3 to 10 carbon atoms or its salt and 1 to 10 w/v % of ammonium sulfate. According to Gion, the foregoing conditions cause the proteins, other than albumin, to precipitate, yielding a protein solution containing the proteins in a concentration of 0.5 to 3 w/v %. The solution is then filtered and the albumin recovered from the supernatant. According to Gion, the organic carboxylic acid suitable for use in his method includes a carboxylic acid, such as caprylic acid, mandelic acid or citric acid, and the physiologically acceptable one such as an alkali metal such as sodium or potassium, or an alkali earth metal such as calcium, etc. This organic carboxylic acid is used in the Gion method at a final concentration of 3-10 mM, preferably 3-5 mM.

U.S. Pat. No. 6,365,395 (to Antoniou, issued Apr. 2, 2002) discloses a process for selectively removing protein aggregates and virus particles from a protein solution in a two-step filtration process, so as to avoid premature plugging of the filtration membranes used in such separations. In a first step, a protein solution is filtered by tangential flow filtration through a cellulosic ultra filtrate membrane at a transmembrane pressure of between about 1 and about 10 psi to produce a first permeate and a retentate stream. Water or aqueous buffer is added to the retentate stream to form an essentially constant volume retentate stream. The first permeate is filtered through a second ultra filtration membrane to retain virus particles at a retention level of at least 3 LRV and to allow passage there through of a protein aggregate free and virus free protein solution.

Notwithstanding the advances made to date, the purification and isolation methods presently available for recovery of albumin and IgG from human plasma generally suffer from relative poor yields, do not readily lend themselves to automation and do not permit the recovery of both albumin and IgG at the same time. Thus, there continues to exist a need to improve the existing purification and isolation methods with respect to yield while preserving the therapeutically active product sought to be recovered.

### OBJECTS OF THE INVENTION

It is the object of this invention to remedy the above as well as related deficiencies in the prior art.

More specifically, it is the principle object of this invention to provide a method for the purification and isolation of therapeutically active proteins from biological fluids, including human plasma, without prior fractionation of the source of such proteins into discrete fractions.

It is another object of this invention to provide a method for the purification and isolation of therapeutically active proteins from plasma, specifically, IgG and albumin, concurrently from human plasma without prior fractionation of the plasma.

It is yet another object of this invention to provide a method for the purification and isolation of therapeutically active proteins, specifically IgG and albumin, at yields previously unattainable.

Additional objects of this invention include the products attainable by the foregoing methods

### SUMMARY OF THE INVENTION

The above and related objects are achieved by providing a method for the concurrent purification and isolation of albumin and an additional protein, such as IgG, from a fluid composition containing up to about 15%, by weight, of blood plasma proteins obtained from a plasma source that has not undergone prior fractionation. [add discussion of use of different source fluids including partially fractionated plasma source]

In one of the preferred embodiments of this invention, the concurrent purification and isolation of a plasma source includes the recovery of albumin and some other plasma component that is associated with albumin. The albumin appears to enhance the stability of the recovered components by providing a physiological supplement to the isolated materials once it has been removed from its native environment. This preference for concurrent purification albumin along with one or more proteins that associate with albumin, permits recovery of relatively unstable plasma components that ordinarily could not be recovered alone, or if capable of independent recovery, were either only obtained in relatively low yields or suffered loss of therapeutic shortly after such independent recovery.

In accordance with this method, the composition is adjusted to a pH of from about 4.5 to about 7.5 with a physiologically acceptable buffer. The composition is then further modified by the addition, relative to said IgG, of a thermal stabilization effective amount of a polyhydric alcohol, wherein the molar concentration of the alcohol within the fluid composition is at concentration in the range of from about 0.1 to about 0.5M; and, by the addition, relative to albumin, of a thermal stabilization effective amount of a carboxylic acid having from about 3 to 10 carbon atoms, or a physiological acceptable sald of said carboxylic acid, wherein said acid and/or said acid salt is present within the fluid composition, relative to the total concentration of albumin, at weight ratio in the range of from about 0.005:1 to aboutn 0.020:1

The resulting fluid composition is thereafter heated to and maintained at a temperature in the range of from about 55 to 75° C. for a period sufficient to effect essentially complete denaturation of proteins other than the albumin and the IgG components of said composition. Generally, heating the fluid composition for at least about 10 hours is sufficient of this purpose. Thereafter, the fluid composition is cooled and the soluble components thereof, the albumin and IgG, isolated from the solids and suspended matter by a combination of filters designed to initially remove the solids and suspended matter from the fluid composition, and thereafter through an array of tangential flow filtration membranes capable of isolation of soluble protein components of said composition by molecular weight.

### DETAILED DESCRIPTION OF THE INVENTION INCLUDING PREFERRED EMBODIMENTS

Where plasma is fractionated in the traditional manner into separate and more manageable, defined fractions as described in Cohn U.S. Pat. No. 2,390,074, (which is hereby incorporated by reference in its entirety), more than 50% of the recoverable therapeutically active proteins are lost or rendered non-recoverable. In order to appreciate the significance of this loss, one need only appreciate that human serum albumin is the most abundant protein in the blood, with a concentration of about 40 to 55 grams per liter of serum. Since the method of this invention does not require fractionation, in excess of 90% of the albumin and other therapeutically active proteins can be recovered.

As shall be evident from the following, the method of this invention, the prior fractionation of a biological fluid into various constituent protein fractions is neither contemplated, and otherwise inconsistent with the objects of this invention because of the adverse impact of such process upon the efficiencies realized by the method of this invention.

Source of Soluble Proteins The source of the soluble proteins sought to be recovery by the method of this invention can be any animal source, or, alternatively, derivative from a culture wherein an organism is engineered to produce soluble proteins of the type found in biological fluids of animals and/or human. Under some circumstances, it may be appropriate to subject the source of soluble proteins to pre-processing for neutralization or removal of a component thereof that is either highly infectious or which simply causes unacceptable interference in the recovery process. For simplicity of understanding ease of explanation, the method of this invention is herein further described within the context of purification and isolation of IgG and albumin from a human plasma.

Processing Materials In addition to the source of soluble proteins referenced above, all of the materials used in the method of this invention are physiologically compatible with the plasma, and otherwise devoid of contaminants and impurities that can evoke an adverse or undesirable response in an individual that would be a candidate for receipt of the protein products of the method of this invention.

The buffers which can be used in the method of this invention, to adjust and maintain the pH of the fluid composition, include any of the well-known buffering system that is thermally stable within the temperature ranges (˜55 to 75° C.) contemplated by the method of this invention; and, which is otherwise physiologically compatible (does not denature proteins) with the objects of this invention. The readily available buffers which satisfy such requirements include the traditional family of phosphate buffers (e.g. TRIS) and natural buffering agents that are sufficiently thermally stable in the processing environment of the method of this invention.

Stabilizers suitable for use in the method of this invention include the physiologically compatible polyhydric alcohols (herein also “polyols”) which are known and available for the thermal stabilization of IgG, such as are described in Fernandes U.S. Pat. No. 4,440,679, which is herein incorporated by reference in its entirety. The term “polyol” as used herein is inclusive of a substance with more than one hydroxyl group (—OH) and includes polyhydric alcohols and carbohydrates such as sugars. It is preferred that the polyol be water miscible, physiologically compatible with the protein, and have a low molecular weight, i.e., a molecular weight less than about 5000. Higher molecular weight polyols, e.g., polysaccharides such as dextrin, starch, glycogen, cellulose, pentosans, pectin, hemicellulose, and the like, are not preferred for use in the present method because they are generally water immiscible and are difficult to separate from the protein composition after pasteurization has been completed. Typical examples of sugars that may be employed in our method are mono-, di-, and trisaccharides such as arabinose, glucose, galactose, fructose, ribose, mannose, rhamnose, sucrose, maltose, raffinose, melezitose, and so forth. Exemplary of polyhydric alcohols or reduced sugars, included within the purview of the invention are erythritol, ribitol, sylitol, sorbitol, mannitol, etc. Also within the compass of the invention are mixtures of polyols and substances that produce a polyol in the presence of water or heat such as hydrates, actonides, or the like.

Compounds that are suitable for the thermal stabilization of the albumin protein, within the processing environment of this invention, include the physiological carboxylic acids, and their physiological acceptable salts, having from 5 to 10 carbon atoms, such as described in Gion U.S. Pat. No. 4,754,019, which is herein incorporated by reference in its entirety. These organic carboxylic acid suitable for use in this invention include the carboxylic acids such as caprylic acid, mandelic acid or citric acid is preferred. The is physiological acceptable salts of such acids include the alkali metal salts, such as sodium or potassium, or an alkali earth metal such as calcium, etc.

Insofar as the process of this invention is directed to the concurrent purification and isolation of dissimilar proteins from a common composition, it is, of course, understood that the compounds selected for the stabilization of, for example, the IgG and, for example, the albumin, must be compatible with one another within the processing environment of this invention. Thus, the selection of stabilizing agents may require some minimum evaluation to insure that each is free from interference or interaction with the other, and thereby free from compromise of the stabilization of the protein component for which it was intended.

Manipulative Steps As above noted and once again emphasized, the method of this invention is suitable for the concurrent purification and isolation of diverse protein fractions from a composition containing a biological fluid. It is understood that the term “purification” is intended to include the thermal inactivation and/or denaturation of endogenous and exogenous materials that can be present in a biological fluid, which if allowed to remain in tact could evoke an immune response or cause and infection in the recipient of the products sought to be recovered from the composition. It is also understood that the term “isolation” is intended to include the physical separation of the soluble components of the composition remaining after removal of particulate matter, and that such isolation is based upon discrimination of products on the basis of molecular weight. It is also understood that the phrase “biological fluid” is intended to include a protein containing liquid that is obtained from a natural or synthetic source wherein the protein components of interest are soluble, along with other endogenous and exogenous materials, in such fluid.

Initially, a biological fluid is obtained and the relative concentration of the protein fraction therein confirmed by simple light scattering or other common analytical techniques. Where the biological fluid comprises the plasma fraction of whole blood, the protein concentration therein is well-known, and no such confirmation necessary. In the case of plasma, sufficient buffer and isotonic saline are added to provide a composition containing from about 1 to about 7%, by weight, protein. Upon preparation of the desired composition, the pH is adjusted to within a range of from 4.5 to about 7.5, and preferably to a pH of 6.5.

The protein stabilizers are then added to this buffered composition. Where IgG is desired to recovered from the composition, a polyhydric alcohol stabilizer is added to the composition in an amount in the range of from about 5 to about 7% weight based upon the volume of the composition. Where albumin is desired to be recovered from the composition, a suitable carboxylic acid stabilizer is added to the composition, relative to total albumin, in a weight ratio relative to total protein in the composition, in the range of from 0.005:1 to about 0.020:1. In each instance, the stabilizers selected for use in this method must be compatible with one another and not otherwise diminish the stability of the proteins sought to be recovered. As noted herein and once again emphasized, the method of this invention can be used in conjunction with other purification processes which desire to avoid the traditional alcohol fractionation processing preliminary to purification and isolation of the proteins of interest.

Upon completion of addition of the stabilizers to the composition, the composition is heated to a temperature in the range of from about 55 to 75° C., and maintained at such temperature for an interval sufficient to effect essentially completed denaturation of unstablized endogenous and exogenous proteins that may also be present in the composition. Generally, maintaining the composition at this denaturation temperature for at least about 10 hours is sufficient to accomplish the intended purpose. The composition is then removed from the source of heating, and allowed to cool to room temperature. After the composition is cooled its is filtered to remove the precipitant that was formed during heating. The manner of removal of such particulate matter can be any common filtration technique intended for separation of particulate matter from the liquid phase of the composition. The soluble composition of the composition are further processed to isolate the suspended matter from the soluble proteins. This can include the passage of the composition through one or a series of membranes of graduated porosity until only the soluble proteins remains in the composition

The soluble components of the composition are now isolated from one another by passage through a series of molecular sieves and thereby the dissolve proteins recovered, based upon their molecular weight. For example, in a purified composition containing IgG and albumin, the IgG and albumin can be isolated from one another by tangential flow filtration through a membrane having a pore size of approximately 300,000 Daltons. The permeate recovered in this manner is further subjected to tangential flow filtration through a second membrane having a pore size of approximately 10,000 Daltons. The permeate recovered at this juncture is subject to High Pressure Liquid Chromatographic analysis. Such analysis indicates that the only protein fractions present in the composition are the monomeric IgG, dimeric IgG and albumin.

When amount of IgG and albumin recovered from a given aliquot of biological fluid is compared to the level of each of these proteins in the biological fluid subjected to the method of this invention, the yield attained is greater than ninety percent (90%).

### EXAMPLES

The Examples that follow further define, describe and illustrate a number of preferred embodiments of this invention. Parts and percentages appearing is such Examples are by weight unless otherwise indicated. All equipment and procedures references in these Examples are understood to be standard or conventional unless indicated to the contrary

### Example I

### (Illustration of Separation of IgG and Albumin, as a Mixture, from Bovine Plasma)

Whole blood, 450 ml, of eleven adult bovines vaccinated against foot and mouth disease virus, were collected by extraction from jugular vein of each animal, using standard blood bags with 50 ml of anticoagulant solution for human blood collection in Blood Bank (Vengelen T., 1996). Each blood bag was centrifuged for blood cellular components separation through Human Blood Bank standard procedures (Vengelen T., 1996) and a total of 2,800 ml of bovine plasma were obtained.

Analysis of sample: A sample of plasma was analyzed for total proteins—5.5 weight percent total proteins and, electrophoresis performed to confirm protein composition—albumin 3.0 weight percent; IgG 1.4 weight percent (Tizard, I. R., 2000) and other endogenous proteins (Dus Santos M. J. et al, 2000).

Separation Isolation of IgG and albumin Fraction: To the approximately 2800 mls of recovered plasma was added 5 weight percent sorbitol and octanoic acid 0.08 mmol, for each gram of albumin. This plasma solution was thereafter heated to 60° C. for 10 hours. The resultant mixture comprises a cloudy liquid having particles (e.g. denatured protein) in suspension. These particles were removed by filtration through a steel mesh, and the remaining suspension thereafter tangential flow filtered with membrane having a pore sizes of 0.45 micrometers, to yielding a clear liquid or aqueous solution. That solution was again filtered through by tangential flow filtration through a membrane having a pore sizes of 300,000 Daltons. The resulting permeate was concentrated by tangential flow filtration with membrane pore sizes of 10,000 Daltons obtaining a product that was analyzed by high performance liquid chromatography (HPLC) verifying the presence of a mixture of IgG and albumin, with at least the 80% of antibodies activity anti foot and mouth virus disease. The yields of the mixtures of IgG and albumin separated from plasma are in function of the following variables: the membrane trade mark selected and the procedures used during tangential flow filtration and dialyses.

### Example II

### (Illustration of Separation of IgG and Albumin, as a Mixture, from Bovine Whole Blood)

To approximately 450 mls fetal bovine whole blood, (containing ˜3.6 g % of total proteins), was added 5 weight percent sorbitol and octanoic acid 0.08 mmol, for each gram of albumin. This fetal bovine whole blood was, thereafter, heated to 60° C. for 10 hours, producing a clear liquid with few clots in suspension. The clots were removed by filtration with steel mesh and the clear liquid, with 3.3 weight percent of total proteins, tangential flow filtered through a membrane pore sizes of 0.1 micrometers, yielding a permeate clear liquid with 2.9 weight percent of total proteins. The resulting permeate was analyzed by high performance liquid chromatography (HPLC), verifying the presence of a mixture of fetal bovine albumin and other proteins.

What is claimed is:

1. In a method for the substantially complete recovery of therapeutically active albumin and IgG fractions from plasma, wherein the plasma is thermally stabilized to prevent denaturation of said therapeutically active albumin and IgG, the improvement comprising:
A. Providing a fluid composition containing from about 5 up to about 15%, by weight, of blood plasma proteins obtained from a plasma source that has not undergone prior fractionation;
B. Adjusting said composition to a pH of from about 4.5 to about 7.5 with a physiologically acceptable buffer;
C. Concurrently stabilizing each of said IgG and said albumin of said composition
(1) by adding to said composition, relative to said IgG, of a thermal stabilization effective amount of a polyhydric alcohol, wherein the molar concentration of said alcohol relative to IgG is in the range of from about 0.05:1 to about 0.1:1, and,
(2) by adding to said composition, relative to said albumin, of a thermal stabilization effective amount of a carboxylic acid having about 3 to about 10 carbon atoms, or the physiologically acceptable salt of said carboxylic acid, wherein said acid and/or said acid salt is present within the fluid composition, relative to the total concentration of albumin, at weight ratio in the range of from about 0.005:1 to about 0.020:1,
D. Heating said composition to a temperature in the range of from about 55 to 75° C. for a period sufficient to effect essentially complete denaturation of proteins other than the albumin and the IgG components of said composition;
E. Isolating the IgG and said albumin components of said composition from solid and suspended matter resulting from Step (D); and
F. Separating said albumin and IgG components of said composition from one another based upon molecular weight, thereby concurrently recovering both albumin and IgG protein fractions

2. The method of claim 1, wherein the polyhydric alcohol is selected from the group consisting essentially of

3. The method of claim 1, wherein the carboxylic acid is selected from the group consisting essentially of

4. The method claim 1, wherein Step (D) includes the heating of the composition at about 60° C. for about ten (10) hours.

5. The method of claim 1 wherein Step (E) includes the isolation of the solid and suspended matter of said composition the IgG and Albumin by filtration.

6. The method of claim 1 wherein Step (F) includes the separation of the IgG and albumin fractions of said composition according to molecular weight by tangential flow of said composition, under pressure, through a membrane array wherein soluble components of each of said IgG and said albumin are concurrently fractionated.

7. A method for recovering albumin and IgG from an aqueous medium in the presence of proteins that are endogenous to said medium and/or proteinacious pathogens that are exogenous to said medium, where said method consists essentially of
A. Providing an aqueous fluid medium comprising a protein mixture of albumin, an IgG fraction and other proteinacious materials, wherein the combined protein concentration of albumin and IgG fraction in said mixture is in the range of from about 2 to about 7 weight percent;
B. Adjusting the pH of said fluid medium to within a range of from about 4.5 to about 7.5 by addition to said medium of a from about 5 to 7 weight percent sorbitol and octanoic acid, or a sodium salt of octanoic acid, the relative concentration of said acid and/or acid salt to albumin being in the range of from about 0.005:1 to about 0.020:1 by weight, so as to stabilize each of said albumin and said IgG against denaturation;
C. Heating said fluid medium to a temperature in the range of from about 55 to 75° C. for a period sufficient to effect essentially complete denaturation of proteins of said mixture, other than said albumin and said IgG components of said composition;
D. Isolating said albumin and said IgG from said fluid medium resulting from Step C; and
F. Separating said albumin and IgG components of said composition from one another based upon molecular weight, thereby concurrently recovering both albumin and IgG protein fractions
